(19) United States
(12) Patent Application Publication
(10) Pub. No.: US 2011/0201992 A1
(43) Pub. Date: Aug. 18, 2011

(54) TAMPON APPLICATOR

(75) Inventors: Steven Smet, Zele (BE); Andreas Matthes, Grosspostwitz (DE)

(73) Assignee: ONTEX HYGIENEARTIKEL DEUTSCHLAND GMBH, Grosspostwitz (DE)

(21) Appl. No.: 13/125,687

(22) PCT Filed: Oct. 23, 2009
(86) PCT No.: PCT/EP2009/063998

§ 371 Date: Apr. 22, 2011

(30) Foreign Application Priority Data
Oct. 24, 2008 (EP) 08167540.7

(51) Int. Cl. A61F 13/26; B29C 45/00; B29C 47/00

(52) U.S. Cl. 604/11; 264/299; 264/209.1

(57) ABSTRACT

A tampon applicator is described which comprises an ejector tube and an outer tube. The outer tube is dimensioned to fit closely and telescopically over the ejector tube. The outer tube has a distal discharge end. The distal end of the ejector tube is formed with one or more radially inwardly slanted fingers. The outer tube can comprise supporting means configured to restrict radially outward expansion of the fingers of the ejector tube when the tampon applicator is in telescopically compacted position.

### FIELD OF THE INVENTION

The present invention relates to tampon applicators and is particularly directed to compact tampon applicators of the type employing telescoping tubes. More specifically, the invention concerns improvements of applicators, wherein the distal end of the ejector (or inner) tube of said applicators is formed with biased fingers.

### BACKGROUND OF THE INVENTION

Most commercially available tampon applicators consist of a pair of telescoping tubes.

In a compact tampon applicator design the ejector (or inner) tube is telescoped into the outer tube while a tampon is stored in the distal end of the ejector tube. Operatively, the ejector tube is “cocked” by being withdrawn proximally out from its stored position in the outer tube (leaving behind the tampon) until the distal end of the ejector tube is positioned to engage the proximal end of the stored tampon. Then the ejector tube can be used as a plunger and its re-insertion into the outer tube expels the tampon through the distal discharge end of the outer tube.

The distal end of the ejector tube is commonly formed with a plurality of fingers separated from one another by slots. Such fingers are typically made so as to be biased towards a closed configuration. Hence, the fingers can slide over the outer surface of the tampon when the ejector tube is being cocked, whereas once the ejector tube has been cocked, the distal edges of the fingers define a circumference smaller than the circumference of the base of the tampon and thus capable of engaging said tampon base.

A frequent cause of malfunction of compact tampon applicators is the re-entry of the tampon into the ejector tube when the latter is pushed back into the outer tube after the cocking step. Hence, instead of engaging the base of the tampon (which is requisite for ejection of the tampon), the fingers of the ejector tube assume or are forced into a more open configuration, whereby the ejector tube simply slides over and once again receives the tampon. Clearly, in such situations, the bias of the fingers of the ejector tube towards a closed configuration has been inadequate.

Previous manners to address the above complication included the use of tampons with a radially more expanded base. Although this improves engagement of the tampon base with the fingers of the ejector tube, it also imposes additional requirements on the manufacture of the tampons and increases the chances of proximal displacement of the tampon during the cocking step. Alternatively, an even stronger propensity towards the closed configuration has been imposed on the fingers of the ejector tube. However, this increases the frictional contact between said fingers and the outer surface of the tampon, resulting in proximal displacement of the tampon and/or scraping of the tampon's surface during the cocking step.

Thus, prior ways to solve the problem of tampon re-entry into the ejector tube have not been completely satisfactory and need to be replaced or complemented by additional manners.

It is therefore an object of the invention to provide a further solution to the problem of tampon re-entry into the ejector tube upon the cocking step, thereby reducing the malfunctioning of compact tampon applicators in use.

### SUMMARY OF THE INVENTION

Having conducted assessments, the present Applicant has realised that the fingers of the ejector tube of compact tampon applicators tend to gradually relax or loosen. In other words, said fingers gradually lose their propensity or bias towards closed configuration and undergo radially outward expansion or opening out.

Such loosening of the fingers of the ejector tube can be pronounced particularly during prolonged storage (e.g., several weeks or months) of the product, which is commonplace both before (e.g., manufacturing, warehouse storage, transport, retailer storage, etc.) and after the product has been purchased by a consumer. Moreover, such loosening may occur more commonly with ejector tube fingers having relatively greater axial length.

The Applicant has contemplated that this gradual loosening or opening out of the fingers of the ejector tube can at least partly underlie the failure of compact tampon applicators, which involves the re-entry of the tampon into the ejector tube during reinsertion of the latter into the outer tube.

The Applicant has next surprisingly realised that if a compact tampon applicator includes supporting means that restricts the radially outward expansion or loosening of the fingers of the ejector tube when the tampon applicator is in a compacted or collapsed position (i.e., the standard configuration in which compact tampon applicators are manufactured, packaged, stored and offered to users), then said fingers can better maintain their bias towards close configuration even during (prolonged) storage. This provides for improved engagement of the fingers with the base of the tampon following cocking of the ejector tube. The present modification of compact applicators therefore improves the reliability of the ejector tube in functioning as a plunger, and reduces the failures of compact applicators due to the re-entry of the tampon into the ejector tube during reinsertion of the latter into the outer tube.

Accordingly, in an aspect the invention provides a tampon applicator comprising an ejector tube and an outer tube dimensioned to fit closely and telescopically over said ejector tube and having a distal discharge end, wherein the distal end of the ejector tube is formed with one or more radially inwardly biased fingers, characterised in that the outer tube comprises supporting means configured to restrict radially outward expansion of the fingers of the ejector tube when the tampon applicator is in telescopically compacted position. Particularly disclosed is such tampon applicator wherein said supporting means comprises or is formed by one or more protrusions on the inner surface of the outer tube, such as for example wherein the supporting means comprises or is formed by a circumferentially extending continuous protrusion or a plurality of circumferentially arranged discrete protrusions on the inner surface of the outer tube.

The Applicant has also realised that the problem of tampon re-entry into the ejector tube during the cocking step may be at least partially attributed to radially outward expansion or enlargement of a tampon disposed in the ejector tube, particularly upon prolonged storage of the tampon applicator assembly. Such tends to occur for example due to gradual relaxation of the compressed material of the tampon. Enlargement of the tampon disposed in the ejector tube may also disadvantageously force the fingers of the ejector tube radially outwardly.

The Applicant has next surprisingly realised that if a compact tampon applicator includes tampon expansion control means configured to restrict the radially outward expansion or enlargement of a tampon disposed in the ejector tube when the tampon applicator is in a compacted or collapsed position, then problems of tampon re-entry into the ejector tube upon cocking can be reduced.

Accordingly, in an aspect the invention provides a tampon applicator comprising an ejector tube and an outer tube dimensioned to fit closely and telescopically over said ejector tube and having a distal discharge end, wherein the distal end of the ejector tube is formed with one or more radially inwardly biased fingers, characterised in that the outer tube comprises tampon expansion control means (in short, “control means”) configured to restrict radially outward enlargement or expansion of a tampon disposed in the ejector tube when the tampon applicator is in telescopically compacted position. Particularly disclosed is such tampon applicator wherein said control means comprises or is formed by one or more protrusions on the inner surface of the outer tube, such as for example wherein the control means comprises or is formed by a circumferentially extending continuous protrusion or a plurality of circumferentially arranged discrete protrusions on the inner surface of the outer tube.

Further aspects of the invention relate to: the outer tube as such of the tampon applicator as taught herein; a tampon applicator assembly comprising the tampon applicator as taught herein and a tampon; a method for manufacturing the above tampon applicator, outer tube or assembly (e.g., an exemplary method may entail a suitable step specifically adapted for forming, such as moulding, said objects); an apparatus specifically designed for manufacturing the above tampon applicator, outer tube or assembly (e.g., an exemplary apparatus may be a suitable moulding apparatus or may comprise moulding means specifically designed for moulding said objects); and use of the above assembly for discharging the tampon.

### BRIEF DESCRIPTION OF FIGURES

The invention will be described in the following in greater detail by way of example only and with reference to the attached drawings of non-limiting embodiments of the invention, in which:

FIG. 1 illustrates a side view of the compact tampon applicator according to an embodiment of the invention in telescopically compacted position.

FIG. 2 illustrates an expanded view of the distal end of the applicator in the compacted position.

FIG. 3 illustrates a top view of the inner surface of the outer tube cross-sectioned along a longitudinal plane.

FIG. 4 illustrates an expanded top view of the inner surface of the outer tube cross-sectioned along a longitudinal plane.

FIG. 5 illustrates various views of an outer tube comprising an embodiment of tampon expansion control means 12 as taught herein.

FIG. 6 illustrates in a) a cross-sectional view through the outer tube of an applicator along the line A-A′ in FIG. 5b; and under b) a corresponding cross-sectional view of a tampon.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, term definitions may be included to better appreciate the teaching of the present invention.

In the figures, the reference sign 1 generally denotes a tampon applicator of the compact type comprising an ejector tube 5 and an outer tube 4 dimensioned to fit closely and telescopically over said ejector tube and having a distal discharge end 2.

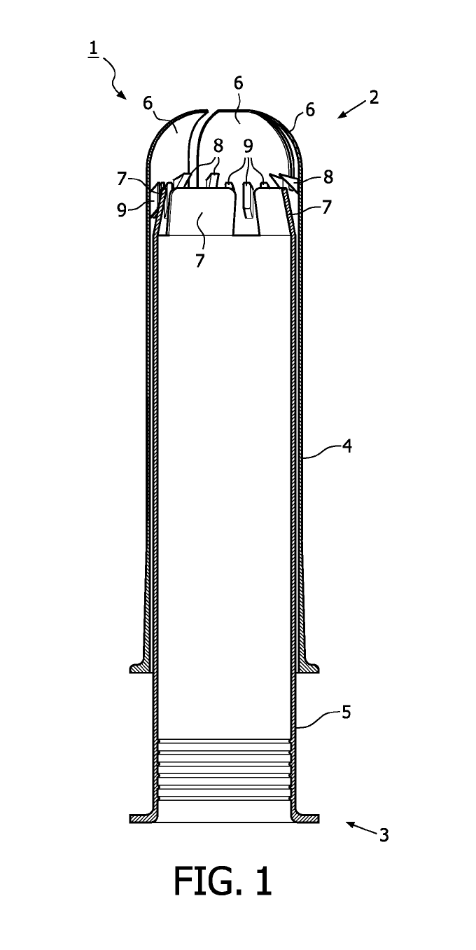

As used herein, the term “proximal end” refers to those portions of the tampon applicator and of its parts that are most remote from the body of a user when a tampon is being emplaced in a body cavity using said applicator. The term “distal end” refers to those portions of the tampon applicator and of its parts that are closest to the body of the user when the tampon is being emplaced. Accordingly, the terms “proximal” or “proximally”, and “distal” or “distally”, as used herein, specify that a given portion or structure of the tampon applicator or of its parts is relatively closer to, respectively, the proximal end or the distal end of the applicator or of its parts. Similarly, the terms “proximal direction” and “distal direction” refer to the directions towards the proximal end or towards the distal end of the tampon applicator or of its parts, respectively. In FIG. 1, the distal and proximal ends of the tampon applicator as meant herein are denoted by reference signs 2 and 3, respectively; and the distal and proximal directions as meant herein are denoted by reference arrows 10 and 11, respectively.

The terms “compact (tampon) applicator” or “(tampon) applicator of the compact type” generally encompass tampon applicators known in the art as such. Typically, compact tampon applicators comprise an inner (or ejector) tube adapted to store a tampon therein and an outer tube dimensioned to fit closely and telescopically over the ejector tube. The outer tube is configured to pass the tampon through an opening in its distal end during an ejection operation. When a tampon applicator assembly comprising a compact tampon applicator and a tampon is prepared for use by a consumer, the tampon is stored in the distal end of the ejector tube and the ejector tube is for its most part telescoped into the outer tube. Operatively, the ejector tube is cocked by being withdrawn in the proximal direction most of the way from its stored position in the outer tube, while the tampon is left behind in the distal end of the outer tube, until the distal end of the ejector tube is positioned to engage the proximal end (or base) of the stored tampon. Once cocked, the ejector tube is used as a hollow plunger for ejecting the tampon positioned within the distal end of the outer tube through the distal discharge opening.

The present invention may be employed with tampon applicators intended for the placement of tampons in any opening, tubing, piping or body cavity. Preferably, the present tampon applicators may be used for placing a tampon into the vaginal cavity of a female mammal, particularly a female human. Hence, the invention in particular concerns catamenial tampon applicators, especially for human use.

Preferably, outer and ejector tubes of compact applicators may be constructed from a suitable smooth plastic material, e.g., polyethylene, using a suitable process, such as, e.g., the tubes may be moulded (e.g., injection moulded) or prepared from extruded tubing.

As shown in the figures, the outer tube 4 and ejector tube 5 are generally cylindrical. The tubes may, however, contain a slight taper towards their respective distal ends, particularly where such taper can assist the discharge of plastic tubes after moulding. The ejector tube 5 is dimensioned to easily slide within the outer tube 4, with minimal clearance in between. The ejector tube 5 is also preferably slightly longer that the outer tube 4 to ensure complete ejection and proper depth of the placement of the tampon. Being hollow, the ejector tube 5 also permits proper placement of a withdrawal string such as commonly provided at the withdrawal end of tampons.

The outer tube 4 may further comprise an outwardly directed circumferential flange at its proximal end. The outer proximal surface of the outer tube 4 may be provided with a ridged or knurled surface, which provides for a firmer grip when this portion of the outer tube 4 is held between fingers.

The ejector tube 5 may also include an outwardly directed circumferential retention flange at its proximal end. The flange controls the extent of insertion of the ejector tube 5 into the outer tube 4 of the applicator. The flange of the ejector tube 5 will abut the retention flange of the outer tube 4 when the ejector tube 5 is fully inserted into the outer tube 4. The inner proximal portion of the ejector tube 5 may optionally be provided with a ridged or knurled surface, which provides for a firmer grip when a finger is inserted into the ejector tube.

Although not illustrated in the figures, the present compact tampon applicators preferably include a restraining means, e.g., an interlocking restraining means, to prevent disassembly of the outer 4 and ejector 5 tubes during the cocking step. Typical restraining means may be formed by circumferentially extending, raised structures (such as, e.g., ring, ribs, rims, flanges, lips or other-shape protrusions) provided on the outer distal surface of the ejector tube 5 and on the inner proximal surface of the outer tube 4. For example, a stopping ring may project radially inwardly from the inner surface of the outer tube 4 near its proximal end and another ring or a plurality of circumferentially aligned protrusions may project radially outwardly from the outer surface of the ejector tube 5 near its distal end (usually disposed proximally adjacent to the base of the fingers 7 of the ejector tube 5). The height of these raised structures is such that they are radially spaced from the opposing surface of the tube other than that on which they are provided when the ejector tube 5 is inserted in the outer tube 4 and when it is being withdrawn in the proximal direction most of the way. However, the structures overlap radially, and will eventually engage when the ejector tube 5 is sufficiently withdrawn from the outer tube 4, thus preventing complete withdrawal and disassembly of the applicator.

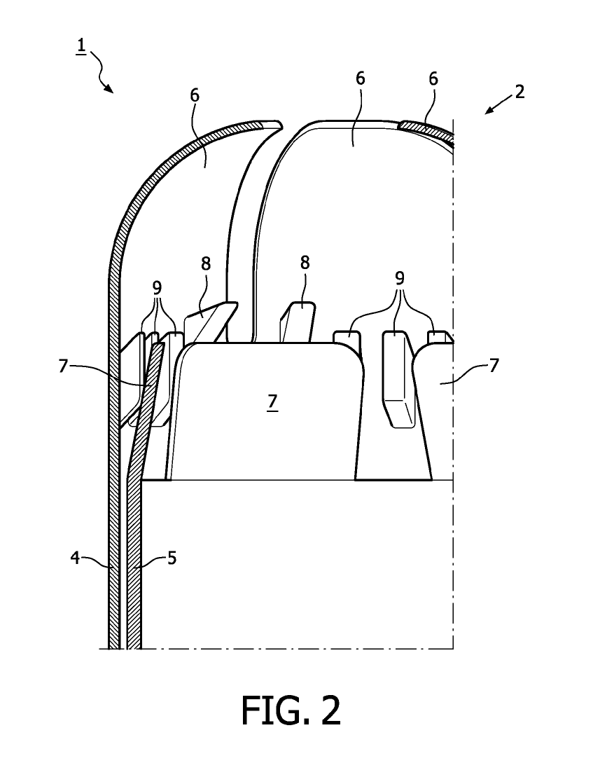

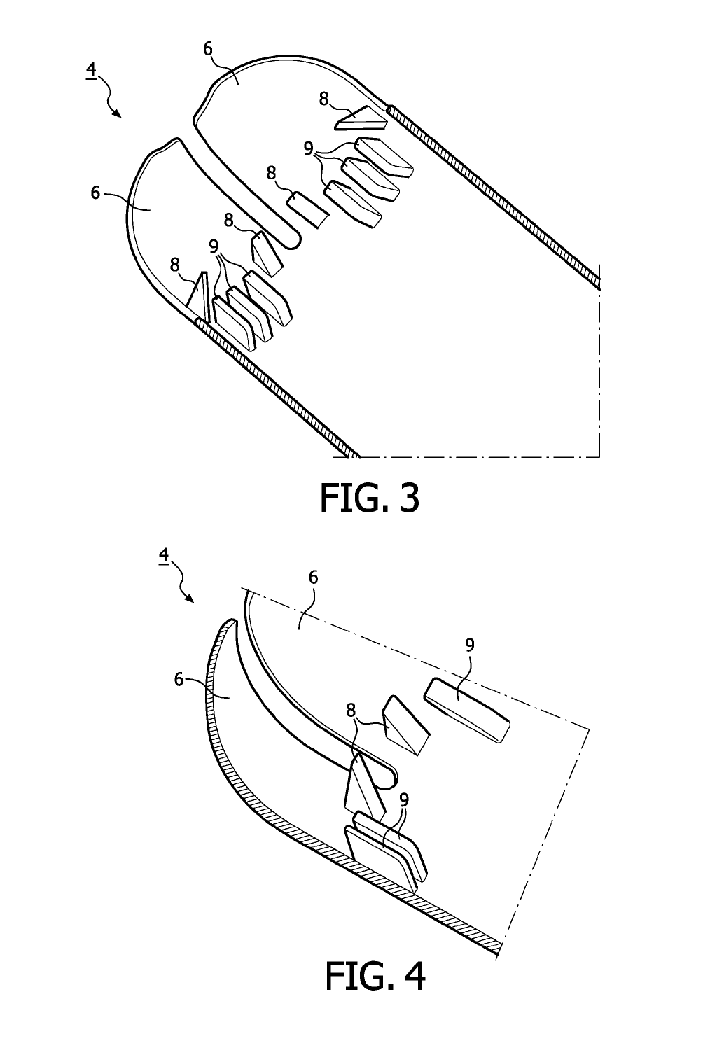

Essentially any restraining means known in the art may be used in the present compact tampon applicators. For example, these encompass any of the restraining means described in EP 0 355 396 B1 on page 9, line 1, through page 11, line 31 under the heading “Earlier Tube Interlocks”, which are herein incorporated by reference. Further, these also encompass the restraining means disclosed in EP 0 355 396 B1, in particular on page 8, line 1 through line 57 under the heading “Improved Tube Interlock”, herein incorporated by reference, which comprises a pair of two raised rings on the inner surface of the outer tube at its proximal end, the respective facing slopes of which defined an interposed valley, and another raised ring-like structure on the outer distal surface of the ejector tube. When the ejector tube is withdrawn from the outer tube in the proximal direction, the ring-like structure on the ejector tube becomes engaged (i.e., interlocked) within the valley on the outer tube, thus preventing the disassembly of the outer and ejector tubes. Yet further, these also encompass the restraining means disclosed in EP 1 695 680 A1, as illustrated in FIGS. 2 and 3 and the accompanying description thereof, herein incorporated by reference, which comprises a circumferentially-extending raised means typically provided on the outer distal surface of the ejector tube and a set of at least three adjacent circumferentially-extending raised rings typically provided on the inner proximal surface of the outer tube. Two of the raised rings define an interposed valley that engages the raised means, while the additional at least one ring provides an extra means of restricting the lateral movement or wobble of the ejector tube relative to the outer tube.

Preferably, the distal discharge end 2 of the outer tube 5 is formed with a plurality, e.g., 2 to 16, more commonly 2 to 8, and most commonly 4 conventional petal sections 6 which are separated from each other by respective slots. The petal sections 6 are made relatively flexible and are normally biased in a substantially arcuate closed configuration to form a rounded tip having a central opening at the distal end 2. The rounded shape of the distal end 2 helps facilitate the insertion of the tampon applicator 1 into a cavity.

Compact tampon applicators commonly include means 8 known per se configured to secure the stored tampon in the distal end of the outer tube 4. As exemplified in the figures, such means may typically include a plurality of, e.g., 2 to 16, more commonly 2 to 8, and most commonly 4 inward protrusions 8 on the inner surface of the outer tube 4, circumferentially arranged approximately at the base of the petal sections 6. The means 8 may alternatively be formed as a continuous raised ring approximately at the base of the petal sections 6, instead of a plurality of inward protrusions 8.

The inward protrusions 8 are configured to engage the insertion end of a tampon stored in the distal end of the ejector tube 5, thereby preventing axial displacement of the tampon in the proximal direction 11 during the cocking of the ejector tube 5. The protrusions 8 may have various shapes known in the art, such as, e.g., pins, ribs, lips or flaps protruding substantially radially inwardly from the inner surface of the outer tube 4, and preferably slanted towards the distal discharge end 2 thereof. The slanting of the protrusions 8 towards the distal end 2 of the outer tube 4 facilitates a secure grip of the tampon, even while not interfering or interfering only minimally with the ejection of the tampon.

The particular means 8 for securing the tampon exemplified in the figures include eight protrusions 8, two disposed at the base of each petal section 6. They display a substantially right triangular profile with the hypotenuse (i.e., the longest side) extending at an acute angle from the inner wall of the outer tube 4 towards the central opening at the distal end 2, one of the catheti (i.e., the shorter sides) facing the distal end of the outer tube 4, and the other cathetus being the contact side with the inner surface of the outer tube 4. The illustrated projections are relatively thin in the circumferential direction, with thickness ranging, e.g., from 0.2 mm to 5 mm, thus saving raw material.

The distal end of the ejector tube 5 of the present tampon applicators is formed with one or more conventional fingers 7. Preferably, the ejector tube 5 may comprise a plurality, e.g., 2 to 16, preferably 2 to 8, more preferably 2 to 6, and most commonly four such fingers 7. The fingers 7 are commonly separated from one another by slots or slits, usually straight longitudinal slots or slits.

The fingers 7 of the ejector tube 5 are made to be biased radially inwardly. Commonly, the fingers 7 may be biased such that their distal edges define an imaginary circumference smaller than the circumference defined by the base of the fingers 7. Hence, the fingers 7 of the ejector tube 5 may be radially inwardly slanted or inclined and/or may display a propensity to slant or incline radially inwardly. The fingers 7 are preferably substantially elastically flexible or bendable, to allow for their opening out when pushed radially outwardly, e.g., when the ejector tube 5 accommodates a tampon or when the ejector tube 5 is being slid over the tampon surface during the cocking step. However, one shall appreciate that the fingers 7 need to be sufficiently firm to adequately engage with the base of the tampon during the ejection step.

When a tampon applicator assembly comprising a compact tampon applicator as taught herein and a tampon is provided to a consumer for use, the ejector tube 5 is for its most part disposed within the outer tube 4 and over a stored tampon (not shown). Generally, the ejector tube 5 may be telescoped into the outer tube 4 sufficiently far to minimise the overall length of the tampon applicator assembly, but not so far as to hamper grasping of the proximal end of the ejector tube 5 by the user or to cause the ejector tube 5 and/or the resident tampon to push open the petal sections 6 of the outer tube 4. The distal edges of the fingers 7 of the ejector tube 5 may axially (“axially” is used throughout in connection to the longitudinal axis of the applicator) abut the inward protrusions 8 of the outer tube 4 configured to engage and secure the tampon, but commonly the fingers 7 do not substantially axially overlap with the inward protrusions 8. Alternatively, the inward protrusions 8 may project through the slots separating the fingers 7 of the ejector tube 5 to engage the tampon, although this last configuration may be less common, since it requires positioning the outer and ejector tubes such that the inward protrusions 8 project through the slots between the fingers 7. The fingers 7 of the ejector tube 5 may be forced radially outward by the carried tampon. This permits the fingers 7, and the remainder of the ejector tube 5, to pass over the tampon that is secured in the outer tube 4 by the inward projections 8 during the cocking of the ejector tube 5. Then, because of the radially inward bias of the fingers 7, when the ejector tube 5 is sufficiently withdrawn, the fingers 7 will incline or flex inwardly to a lesser diameter than that of the base of the tampon. Consequently, when the ejector tube 5 is axially moved in the distal direction 10, the tampon will be engaged at its proximal end by the fingers 7 and urged out the distal discharge end 2 of the outer tube 4. Any compact tampon applicator known in the art of essentially the above type is suitable for being improved according to the present invention.

The above described configuration of the tampon applicator, as also illustrated in FIGS. 1 and 2 (not depicting a resident tampon), i.e., with the ejector tube 5 telescoped into the outer tube 4 to an extent as generally employed in the art for compact tampon applicators when being provided to users, is commonly referred to as telescopically “compacted” or “collapsed” position of the tampon applicator.

The outer tube 4 of the applicator of the present invention may comprise supporting means 9 configured to restrict radially outward expansion of the fingers 7 of the ejector tube 5 when the tampon applicator is in telescopically compacted position. Thereby, the gradual loosening or opening out of the fingers 7 of the ejector tube 4, such as would otherwise occur during (prolonged) storage of tampon applicators or assemblies, is counteracted. Consequently, the radially inward bias of the fingers 7 is effectively maintained, and the fingers 7 can more adequately engage the base of the tampon once the ejector tube 5 has been cocked.

As used herein, the term “restrict” with reference to radially outward expansion of the fingers 7 encompasses situations in which such expansion or relaxation of the fingers 7 is only partly prevented by the supporting means 9 (e.g., where the fingers are allowed to expand to a certain extent and not further when the applicator is in compacted position), as well as situations in which the expansion or relaxation of the fingers 7 is completely prevented by the supporting means 9 (i.e., where the fingers 7 are not allowed any extent of radial expansion when the applicator is in compacted position).

In an embodiment, the supporting means 9 may be configured to engage the fingers 7 of the ejector tube 5 when the tampon applicator 1 is in telescopically compacted position. A non-limiting example of such engagement is shown in FIGS. 1 and 2. Hence, the supporting means 9 is configured to make contact with the fingers 7, whereby the supporting means 9 can hold the fingers 7 in their initial position and can substantially completely prevent any expansion or opening out of the fingers 7. This configuration is particularly effective at maintaining the radially inward bias of the fingers 7.

In a further improvement the supporting means 9 is configured to exert a force having radially inward component on the fingers 7 of the ejector tube 5 when the tampon applicator 1 is in telescopically compacted position. Hence, the supporting means 9 not only restricts the loosening of the fingers 7, but helps to maintain or even increase or strengthen the advantageous radially inward bias of the fingers 7, by pushing or forcing said fingers towards a more closed configuration, i.e., exerting a radially inward push or force (or force comprising a radially inward component) on the fingers 7.

By means of example and not limitation, when engaged by the supporting means 9, the inclination of the fingers 7 vis-à-vis the longitudinal axis of the ejector tube 5 may be between about 2° and about 45°, e.g., between about 5° and about 15° (i.e., the angle between the fingers 7 and a plane perpendicularly transverse to the body of the ejector tube 5 may be between about 45° and about 88°, e.g., between about 75° and about) 85°).

In an embodiment, when the ejector tube 5 comprises two or more fingers 7, each of said fingers 7 may be preferably supported by the supporting means 9. Moreover, one or more supporting means 9 may be provided for supporting each of the fingers 7.

In an embodiment, the supporting means may comprise one or more raised means 9 provided on the inner surface of the outer tube 4. The term “raised” in this connection refers to any element or structure raised (i.e., raising or projecting radially inwardly) relative to the inner surface of the outer tube 4. Such raised means 9 represents a relatively undemanding manner of achieving the desired function of the supporting means. For example, the raised means 9 may be integral to the outer tube, and may be easily achieved, e.g., by suitably moulding the outer tube to provide the desired profile on its inner surface.

Accordingly, in an embodiment the raised means 9 is formed by a circumferentially extending continuous protrusion (e.g., a ring or a ring-like protrusion) or, as illustrated in the figures, by a plurality of circumferentially arranged discrete protrusions 9 on the inner surface of the outer tube 4. Any or each of the fingers 7 may be supported by one or more than one such protrusions 9, e.g., without limitation by between 1 and 10, or between 2 and 6, or between 3 and 5 such protrusions 9. Such protrusion may have any shape compatible with their purpose, such as, e.g., pins, ribs, lips, flaps, rods, hemispheres, cylinders, etc. protruding substantially radially inwardly from the inner surface of the outer tube 4.

The present Applicant has further realised that the radially outward expansion of the fingers 7 of the of the ejector tube 5 may also occur as a consequence of radially outward expansion or enlargement of a tampon disposed in the ejector tube 5, particularly upon prolonged storage of the tampon applicator assembly. The width-wise enlargement of the tampon disposed in the ejector tube 5 may thus force the fingers 7 radially outwardly, thereby causing or further contributing to the loosening of said fingers 7.

The Applicant has thus contemplated that the loosening or relaxation of the fingers 7 of the ejector tube 5 may also be restricted or prevented indirectly via restricting or preventing the radially outward expansion of the tampon disposed in the ejector tube 5.

Accordingly, in an embodiment the supporting means 9, such as particularly the protrusion or protrusions 9, may be configured to engage a tampon disposed in the ejector tube 5 of the tampon applicator 1 when the tampon applicator 1 is in telescopically compacted position. Hereby, the supporting means 9 can make contact with the tampon and can restrict or prevent its width-wise expansion, and thereby restrict or prevent radially outward opening of the fingers 7. This configuration is particularly effective at maintaining the radially inward bias of the fingers 7.

In another embodiment, the supporting means 9, such as particularly the protrusion or protrusions 9, may be configured to exert a force having radially inward component on a tampon disposed in the ejector tube 5 of the tampon applicator 1 when the tampon applicator 1 is in telescopically compacted position. Hereby, the supporting means 9 can help to maintain the radial dimensions of the tampon within desirable limits, and thereby can restrict or prevent radially outward opening of the fingers 7. This configuration is particularly effective at maintaining the radially inward bias of the fingers 7.

It shall be appreciated that the supporting means may comprise members, such as one or more protrusions 9, which directly engage with and/or exert a radially inward force on the fingers 7, as well as members, such as one or more protrusions 9, which engage with and/or exert a radially inward force on the tampon, thereby indirectly restricting or preventing the loosening of the fingers 7.

Further characteristics of supporting means configured to act on the tampon are explained elsewhere in this specification, particularly in connection with tampon expansion control means.

The supporting means or protrusions 9 may engage or contact the fingers 7 at various axial points of the fingers 7 (e.g., adjacent to the base of the fingers 7 and/or adjacent to the distal edge of the fingers 7 and/or at one or more points between said base and said distal edge of the fingers 7). Supporting means or protrusions 9 which should engage close to the base of the fingers 7 can be relatively less raised than supporting means or protrusions 9 that should engage close to the distal edge of the fingers 7. Relatively less raised protrusions 9 may cause less obstruction when a tampon is being ejected. Protrusions 9 engaging close to the distal edge of the fingers 7 need to exert less force to achieve a given degree of inclination of the fingers 7. Thus, the fingers 7 will show less counterforce, making the ejector tube 5 more stable in its telescoped position. Mixed advantages are achieved by supporting means or protrusions 9 that engage between the base and the distal edge of the fingers 7.

In a further embodiment, the protrusion or protrusions 9 may themselves comprise a slope raising in the axially distal direction 10. Such protrusions can achieve a gradual and smooth contact or engagement with the fingers 7 when the ejector tube 5 is being telescoped into the outer tube 4, such as to bring the applicator in its telescopically compacted position. Moreover, protrusions 9 with such slope can cause less obstruction when a tampon is being ejected in the distal direction 10. It shall be appreciated that the latter effect may also be more generally achieved by protrusions 9 whose edges, in particular whose proximal edges, are substantially rounded.

In a further embodiment, at least a portion of said slope of the protrusions 9 raising in the axially distal direction 10 may match at least a portion of the slope of the biased fingers 7 of the ejector tube when the tampon applicator 1 is in telescopically compacted position. This can achieve particularly effective engagement of the protrusions 9 and the fingers 7 mediated by their compatible surfaces.

As can be understood from the above explanation, the raised means or protrusions 9 should support the fingers 7 of the ejector tube 5 when the applicator is in compacted position. Hence, the raised means or protrusions 9 are preferably disposed in the area of the inner surface of the outer tube 4 which axially overlaps with said fingers 7 when the applicator is compacted. Expectedly, the raised means or protrusions 9 will thus be provided relatively close to the distal end of the outer tube 4.

Preferably, the raised means or protrusions 9 may be provided on the inner surface of the outer tube 4 proximally adjacent to the base of the petal sections 6. Typically, in compacted applicators the distal fingers 7 of the ejector tube 5 will not substantially project into the dome formed by the petal sections 6 of the outer tube 4. Hence, disposing the raised means or protrusions 9 just proximally to the base of the petal sections 6 (e.g., as indicated in FIG. 3) generally allows the protrusions 9 to come into contact with the fingers 7 of the ejector tube in the compacted applicator.

Another advantage of the above placement of the protrusions 9 (e.g., vis-à-vis placement on the petal sections 6 themselves) is that, compared to the rather bendable petal sections 6, the portion of the outer tube 4 proximally adjacent to the base of the petal sections 6 can better withstand any forces exerted thereon by the fingers 7 of the ejector tube 5 without undesirable deformation.

Hence, in an embodiment the raised means or protrusions 9 may be provided on the inner surface of the outer tube 4 proximally adjacent to the base of the petal sections 6 and within an axial distance from the base of the petal sections 6 substantially equal to the axial length of the fingers 7 of the ejector tube 5. Such placement can ensure good axial overlap between the protrusions 9 and the position of the fingers 7 of the ejector tube 5 in a compacted applicator, thereby facilitating their engagement. Nevertheless, an even more proximal placement of the protrusions 9 within the outer tube 4 can be envisaged.

As explained, the inner surface of the outer tube 4 may also comprise protrusions 8 for securing the tampon, usually disposed at the base of the petal sections 6. In embodiment, the raised means or protrusions 9 may be disposed proximally relative to said protrusions 8. In another embodiment, the raised means or protrusions 9 may represent separate structures from said protrusions 8. In yet another embodiment, the raised means or protrusions 9 and the protrusions 8 may form parts of a same radially inwardly raised structure or element. Hence, the latter structure or element can advantageously perform both functions. In an embodiment, the radial height of the protrusions 8 may be greater than the radial height of the raised means or protrusions 9.

As noted, the outer tube 4 of the applicator of the present invention may comprise tampon expansion control means 12 configured to restrict radially outward enlargement or expansion of a tampon disposed in the ejector tube when the tampon applicator is in telescopically compacted position.

As used herein, the term “restrict” with reference to radially outward enlargement or expansion of the tampon encompasses situations in which such enlargement or expansion of the tampon is only partly prevented by the control means 12 (e.g., where the tampon is allowed to expand to a certain extent and not further when the applicator is in compacted position), as well as situations in which the enlargement or expansion of the tampon is completely prevented by the control means 12 (i.e., where the tampon, or particularly portions thereof in axial alignment with the control means 12, is not allowed any extent of radial enlargement or expansion when the applicator is in compacted position).

In an embodiment, the control means 12 may be configured to engage the tampon disposed in the ejector tube 5 when the tampon applicator 1 is in telescopically compacted position. Hereby, the control means 12 can make contact with the tampon and can restrict or prevent its width-wise expansion.

In a further improvement the control means 12 is configured to exert a force having radially inward component on a tampon disposed in the ejector tube 5 when the tampon applicator 1 is in telescopically compacted position. Hereby, the control means 12 can help to maintain the radial dimensions of the tampon within desirable limits.

Generally, the control means 12 may engage parts of the tampon which are accessible to elements of the outer tube 4 when the tampon applicator 1 is in compacted position. Such parts of the tampon may particularly but without limitation include the tampon portion which protrudes axially through the distal opening of the ejector tube 5, and the portions which align axially with (i.e., are positioned underneath) the slots between fingers 7 of the ejector tube 5.

In an embodiment, the control means 12 may comprise or be formed by one or more protrusions provided on the inner surface of the outer tube 4. The protrusion(s) of the control means 12 are raised, i.e., they are elements or structures projecting radially inwardly relative to the inner surface of the outer tube 4. The protrusion(s) of the control means 12 may be integral to the outer tube, and may be easily achieved, e.g., by suitably moulding the outer tube to provide the desired profile on its inner surface.

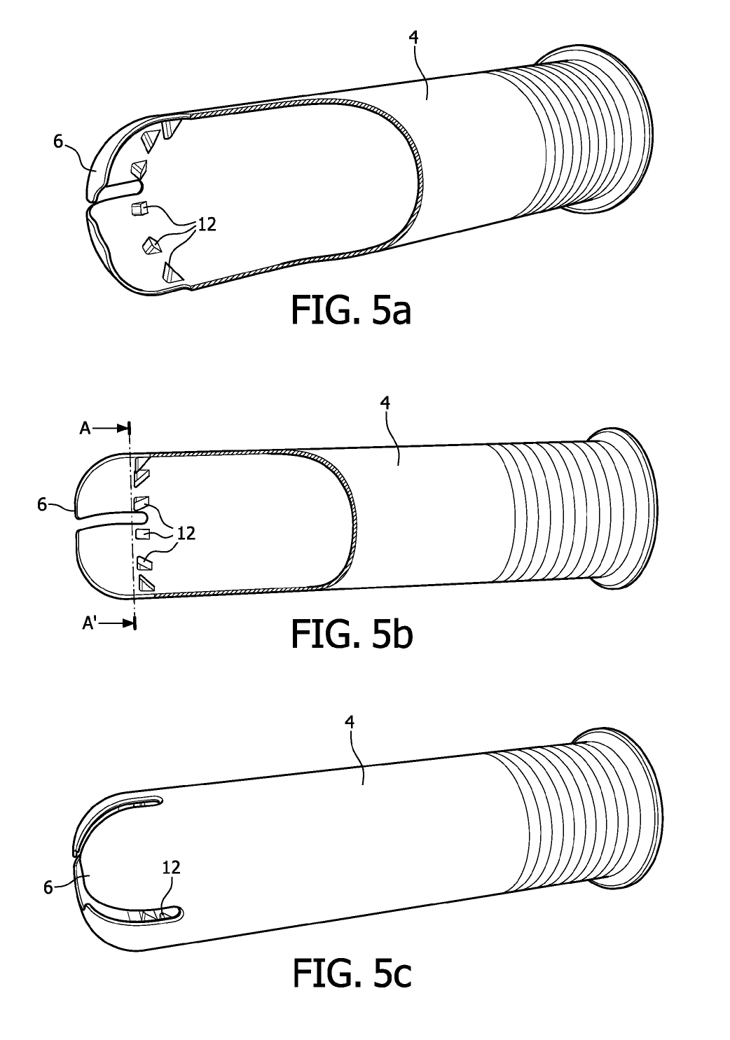

In an embodiment the control means 12 may comprise or be formed by a circumferentially extending continuous protrusion (e.g., a ring or a ring-like protrusion) or, as illustrated in FIG. 5, by a plurality of circumferentially arranged discrete protrusions on the inner surface of the outer tube 4. For example, the control means 12 may comprise or be formed by 4 or more such circumferentially arranged protrusions. Preferably, to maximise the engagement between the control means 12 and the tampon, the control means 12 may comprise or be formed by ≧6, more preferably ≧8, even more preferably ≧10, yet more preferably ≧12 circumferentially arranged protrusions (FIG. 5), or may even comprise or be formed by ≧16 or ≧24 such protrusions. Typically, the control means 12 may comprise less than 40, more preferably less than 30 protrusions.

To provide for comparably homogeneous prevention of tampon enlargement, the protrusions may be spaced at regular circumferential intervals over 360°.

Further, to maximise the prevention of tampon enlargement, the protrusions may engage the tampon over ≧5% or ≧10% of the tampon's circumference (as best viewed in a in suitable cross-section through the tampon applicator comprising a tampon in compacted position), and preferably over ≧20%, such as ≧30%, and more preferably over ≧40% of the tampon's circumference.

The protrusions of the control means 12 may have any shape compatible with their purpose, such as, e.g., pins, ribs, lips, flaps, rods, hemispheres, cylinders, etc. protruding substantially radially inwardly from the inner surface of the outer tube 4. Preferably, the protrusions of the control means 12 may be slanted (i.e., biased to ascend) towards their distal end, as shown in FIG. 5. The slanting of the protrusions towards their distal end 2 facilitates engagement of the tampon, even while not interfering or interfering only minimally with the ejection of the tampon. The latter effect may also be more generally achieved by protrusions whose edges, in particular whose proximal edges, are substantially rounded.

As can be understood from the above explanation, the protrusions of the control means 12 are configured to engage a tampon disposed in the ejector tube 5 when the applicator is in compacted position. To this end, said protrusions 12 may be preferably disposed in the area of the inner surface of the outer tube 4 which is axially distal to the distal edges of the fingers 7 of the ejector tube 5 when the applicator 1 is compacted (hereby, the protrusions 12 are capable of engaging the portion of the tampon projecting distally from the distal end of the ejector tube 5 when the applicator is in compacted position) and/or said protrusions 12 may be preferably disposed in the area of the inner surface of the outer tube 4 which axially overlaps with the slots between the fingers 7 of the ejector tube 5 when the applicator 1 is compacted, such that the protrusions 12 are capable of projecting through said slots and engage the portion of the tampon underneath said slots).

For example, the protrusions 12 of the control means may be provided on the inner surface of the outer tube 4 adjacent (e.g., distally or proximally adjacent) to the base of the petal sections 6. Typically, in compacted applicators the distal fingers 7 of the ejector tube 5 will not substantially project into the dome formed by the petal sections 6 of the outer tube 4. Hence, disposing the protrusions 12 adjacent to the base of the petal sections 6 (e.g., as indicated in FIG. 5) generally allows the protrusions 12 to come into contact with the portion of the tampon projecting through the distal end of the ejector tube 5.

It shall be further understood that protrusions of the control means 12 that are configured to engage a tampon through the slots between the fingers 7 of the ejector tube 5 may be typically provided on the inner surface of the outer tube 4 proximally adjacent to the base of the petal sections 6 and within an axial distance from the base of the petal sections 6 substantially equal to the axial length of the fingers 7 of the ejector tube 5.

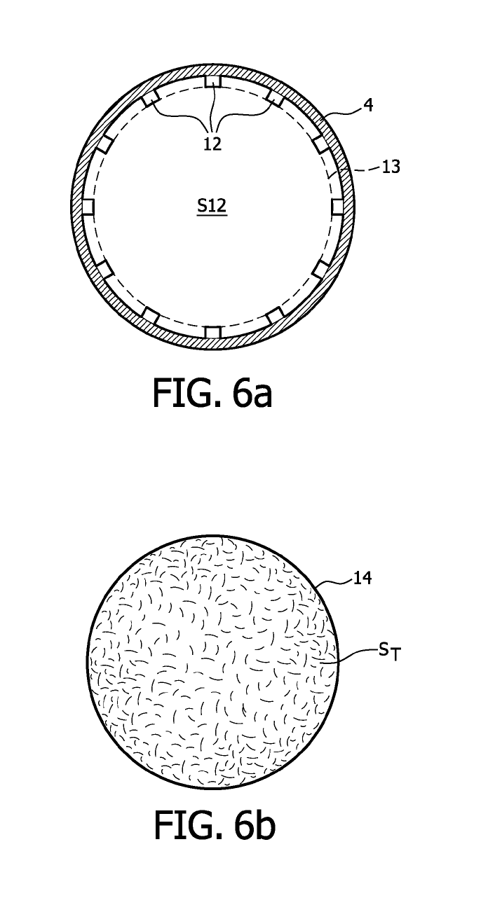

Further, the following feature may be preferred to maximise the restraining effect of the control means 12 on the tampon enlargement (see FIG. 6):—a notional inscribed closed geometry 13 is defined by the radially inward edges of the protrusions of the control means 12 and the surface area of said geometry is denoted S₁₂;—a notional closed geometry 14 is defined by the circumference of a tampon in the axial portion of the tampon to be engaged by said protrusions of the control means 12, and the surface of this geometry is denoted S_{T};—and S₁₂ is equal or smaller than S_{T}, preferably but without limitation S₁₂≦about 97% of S_{T}; or S₁₂≦about 95% of S_{T}; or S₁₂≦about 90% of S_{T}; or S₁₂≦about 85% of S_{T}; or S₁₂≦about 80% of S_{T}; or S₁₂≦about 75% of S_{T}.

And in a specific example, said notional inscribed closed geometry 13 may be the largest circle enclosable by the radially inward edges of the protrusions of the control means 12, and said notional geometry 14 may be the smallest circle circumscribing and enclosing the circumference of the tampon, and the diameter of the circle 13 is equal or smaller than the diameter of the circle 14, preferably but without limitation the diameter of the circle 13 is ≦ about 97% or ≦ about 95% or ≦ about 90% or ≦ about 85% or ≦ about 80% or ≦ about 75% of the diameter of the circle 14.

As explained, the inner surface of the outer tube 4 may also comprise protrusions 8 for securing the tampon, usually disposed at the base of the petal sections 6. In embodiment, the control means 12 may be disposed proximally relative to said protrusions 8. In another embodiment, the control means 12 may represent separate structures from said protrusions 8. In yet another embodiment, the control means 12 and the protrusions 8 may form parts of a same radially inwardly raised structure or element. Hence, the latter structure or element can advantageously perform both functions, i.e., restrict the expansion or enlargement of the tampon and prevent the proximal displacement of the tampon during the cocking of the applicator. In an embodiment, the radial height of the control means 12 may be greater than the radial height commonly used for protrusions 8.

Tampons for use with compact applicators may commonly comprise an enlarged (e.g., mushroom-like) head.

Another tampon for use with the applicator of the invention may have an enlarged (e.g., preferably mushroom-shaped) head, a barrel shape and/or a conical withdrawal end. The shape is reflected in the longitudinal cross-section of the tampon. The enlarged or mushroom head may be, e.g., quasi-spherical, or may be axially prolonged or flattened. The (slightly) enlarged (widened) distal head secures the tampon against proximal displacement. The tampon may further be provided with a conical withdrawal end. The conical shape is one which is preferably truncated from its point. Such conical end guides the tampon during withdrawal, so making withdrawal easier.

Typically, to hold the tampon in position during cocking, protrusions 8 may be configured to rest against and engage with the base of the enlarged head of the tampon, but these usually do not engage with the more proximal parts (body) of the tampon.

In an embodiment, the control means 12 may be advantageously configured to engage with said body of the tampon relatively proximal to the enlarged head. For example, the control means 12 may be configured to engage with areas of the body of the tampon accessible underneath the slots between the fingers 7 of the ejector tube 5. Hence, the protrusions of the control means 12 may be provided on the inner surface of the outer tube 4 and configured to engage the body of a tampon having an enlarged head within an axial distance from the base of said enlarged head substantially equal to the axial length of the fingers 7 of the ejector tube 5. This arrangement can advantageously restrict or prevent the expansion or enlargement of the corresponding portions of the body of the tampon, thereby also restricting or preventing loosening of fingers 7 of the ejector tube 5, which fingers may commonly rest against the body of the tampon proximally adjacent to the head of the tampon in compact tampon applicators.

The present tampon applicator may be used in conjunction with any tampons, in particular catamenial tampons, known in the art.

Tampons may be manufactured as known per se. Typically, a suitable tampon, in particular a self-sustaining shape type tampon, may be made by folding or rolling or otherwise manipulating a strip of absorbent material, such as, e.g., a loosely associated fibrous web, into an elongated shape, e.g., a generally cylindrical shape, known as a “tampon blank” or “softwind” and compressing the softwind into the tampon's ultimate shape. As used herein, “compression” refers to the process of pressing, squeezing, compacting or otherwise manipulating the size, shape, and/or volume of a material to obtain a tampon having a self-sustaining, insertable shape. Usually, said compression or shaping may comprise at least a radial component, i.e., a component of circumferential compression directed towards the central longitudinal axis of the tampon. By means of example, the compression or shaping may be in any or all of the width direction, the radial direction, and the axial direction. A withdrawal string can be attached either to the softwind before compression or following compression to the resulting tampon. An outer cover (e.g., an apertured plastic film or non-woven) may be preferably provided on the softwind before compression, such that it is compressed simultaneously with the absorbent material to the final tampon shape. Alternatively, an outer cover may be secured to the absorbent core following compression.

Preferably, the surface of a suitable tampon may comprise at least one area of radial depression, more preferably may comprise pressed longitudinal grooves. As taught in WO 2006/099944, such surface depressions can reduce contact of the tampon surface with the inner surfaces of the ejector and/or outer tube, and thereby reduce tampon surface scraping during the cocking and/or ejection steps.

In a cross-section through a tampon comprising groove(s), a groove defines adjacent outer surfaces forming ribs flanking the groove. The provision of tampons with pressed grooves and ribs may also increase the absorption and expansion capacity of tampons.

Preferably, the grooves may be longitudinal, i.e., extending along at least a portion of the length of the tampon. In the broadest meaning, a longitudinal groove may be any which, in the direction of its length, is not perpendicular to the longitudinal axis of the tampon. Preferably, the longitudinal grooves may be generally parallel to the central longitudinal axis of the tampon. Preferably, the longitudinal grooves may extend along at least 50%, e.g., at least 60%, preferably at least 70%, e.g., at least 80%, more preferably at least 90%, e.g., at least 95% or even about 100% of the axial length of the elongated surface of the tampon. They may at least partly extend into the insertion and/or withdrawal ends of the tampon.

The grooves and thereby defined ribs may assume various overall shapes in the axial direction, e.g., they may be straight (linear), straight, sinusoidal, meandering, spiral, helical, hyperbolic, or the like. A groove may extend over various portions of the tampon circumference. For example, a groove may extend over at least about 10°, 20°, 30°, 40°, 50°, 60°, 70°, 80°, 90°, 100°, 120°, 130°, 140°, 150° or over more than 150° of the tampon circumference, e.g., over 80° to 120°, or 80° to 150°.

A tampon may comprise varying number (even or odd) of such longitudinal grooves and ribs, e.g., depending on the diameter of the tampon and/or the type of absorption material, etc. Preferably, a tampon may comprise between 3 and 12 grooves and/or ribs, e.g., 3, 4, 5, 6, 7, 8, 9, 10, 11 or 12, more preferably between 6 and 12, even more preferably at least about 8 grooves or ribs, e.g., 8 grooves and/or ribs. Preferably, where the tampon comprises two or more longitudinal grooves, these may be spaced circumferentially at regular intervals, i.e., at regular circumferential angle intervals.

Typically, tampons comprising grooves (and adjacent ribs) can be formed by simultaneous pressing of narrow, strip-shaped sections of the circumferential surface of a tampon blank arranged in a spaced manner to form a number of grooves, preferably longitudinal grooves. The direction of pressing of a groove comprises a radial component of compression, i.e., a component converging toward the central longitudinal axis of the tampon along a radial line, but may also comprise a component of compression diverging from the central longitudinal axis of the tampon (for example, see tampons disclosed in EP 1 547 555, incorporated by reference herein).

Said pressing of the tampon blank produces a highly compressed, solid central part of the tampon, which is typically generally cylindrical with its circumference demarcated by the valley surface of the pressed grooves, and relatively uncompressed ribs interposed between the grooves and extending outwardly from the highly compressed central part. The outer ends of the ribs may but need not be subsequently pressed to provide a soft, smooth circumferential surface of the tampon, while preserving the relatively uncompressed structure of the ribs, achieving good absorption.

Tampons comprising longitudinal grooves and ribs and manners of producing such are disclosed, for example, in DE 3,934,153 C2, U.S. Pat. No. 5,911,712, WO 02/078586, EP 1 108 408, EP 1 459 720 or U.S. Ser. No. 03/208,180, incorporated by reference herein.

A tampon of the invention may optionally include further features, especially features commonly employed with tampons in the art, such as, e.g., one or more surface markings, e.g., printed and/or embossed markings; one or more colours, e.g., applied by printing or impregnation; one or more chemical indicators capable of indicative colour change, e.g., for detecting and indicating medical condition of the user (such exemplary features are disclosed, e.g., in, WO 2006/099944 A1, p. 21, l. 6 to 30, herein incorporated by reference).

Tampons for use with the applicator may comprise known modifications which may improve the working of the telescoping tubes type of applicators. In a non-limiting example, the base of the tampon may be uniquely formed with a concave configuration leaving a more dense centre and a relatively softer peripheral ridge. The softer ridge may aid the proximal end of the tampon in catching on the fingers 7, thus serving as an unidirectional lock to further prevent the tampon from re-entering the ejector tube 5 during expulsion. In another example, the tampon may be a two-diameter tampon. In an example, the tampon may comprise a slightly enlarged head to better secure the tampon against axial displacement.

The liquid-absorbent core of the tampon comprises, consist essentially of or consist of absorbent, preferably hydrophilic material(s), representative, non-limiting examples of which include cellulosic materials, such as rayon, cotton, wood pulp, comminute wood pulp, creped cellulose wadding, tissue wraps and laminates, peat moss, and chemically stiffened, modified, or cross-linked cellulosic fibres; polymeric materials, such as polyester fibres, polyolefin fibres, absorbent foams, e.g., resilient polyurethane foam, absorbent sponges, super-absorbent polymers, absorbent gelling materials; formed fibres, such as capillary channel fibres and multi-limbed fibres; combination materials, such as, e.g., synthetic fibres and wood pulp, including co-formed fibrous structures (e.g., those materials described in U.S. Pat. No. 4,100,324); or any equivalent materials, or combinations or mixtures of above materials.

It is apparent that there has been provided in accordance with the invention, a tampon applicator that provides for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

1. A tampon applicator comprising an ejector tube and an outer tube dimensioned to fit closely and telescopically over said ejector tube and having a distal discharge end, wherein the distal end of the ejector tube is formed with one or more radially inwardly biased fingers, and wherein the outer tube comprises supporting means configured to restrict radially outward expansion of the fingers of the ejector tube when the tampon applicator is in telescopically compacted position, characterised in that said supporting means comprises one or more protrusions on the inner surface of the outer tube.

2. The tampon applicator according to claim 1, wherein the supporting means comprises a circumferentially extending continuous protrusion or a plurality of circumferentially arranged discrete protrusions on the inner surface of the outer tube.

3. The tampon applicator according to claim 1, wherein the protrusion or protrusions are configured to engage the fingers of the ejector tube when the tampon applicator is in telescopically compacted position.

4. The tampon applicator according to claim 1, wherein the protrusion or protrusions are configured to exert a force having radially inward component on the fingers of the ejector tube when the tampon applicator is in telescopically compacted position.

5. The tampon applicator according to claim 1, wherein the protrusion or protrusions comprise a slope raising in the distal direction.

6. The tampon applicator according to claim 5, so configured that at least a portion of said slope of the protrusion or protrusions matches at least a portion of the slope of the biased fingers of the ejector tube when the tampon applicator is in telescopically compacted position.

7. The tampon applicator according to claim 1, wherein the distal discharge end of the outer tube is formed with a plurality of petal sections, and wherein the protrusion or protrusions are provided on the inner surface of the outer tube proximally adjacent to the base of the petal sections.

8. The tampon applicator according to claim 7, wherein the protrusion or protrusions are provided on the inner surface of the outer tube within an axial distance from the base of the petal sections substantially equal to the axial length of the fingers of the ejector tube.

9. The tampon applicator according to claim 1, wherein the protrusion or protrusions are configured to engage a tampon disposed in the ejector tube of the tampon applicator when the tampon applicator is in telescopically compacted position.

10. The tampon applicator according to claim 1, wherein the protrusion or protrusions are configured to exert a force having radially inward component on a tampon disposed in the ejector tube of the tampon applicator when the tampon applicator is in telescopically compacted position.

11. An outer tube for a tampon applicator, the outer tube dimensioned to fit closely and telescopically over an ejector tube for a tampon applicator, a distal end of the ejector tube is formed with one or more radially inwardly biased fingers, the outer tube having a distal discharge end, and wherein the outer tube comprises supporting means configured to restrict radially outward expansion of the fingers of the ejector tube when the tampon applicator is in telescopically compacted position, characterised in that said supporting means comprises one or more protrusions on the inner surface of the outer tube.

12. A tampon applicator assembly comprising a tampon applicator comprising an ejector tube and an outer tube dimensioned to fit closely and telescopically over said ejector tube and having a distal discharge end, wherein the distal end of the ejector tube is formed with one or more radially inwardly biased fingers, and wherein the outer tube comprises supporting means configured to restrict radially outward expansion of the fingers of the ejector tube when the tampon applicator is in telescopically compacted position, characterised in that said supporting means comprises one or more protrusions on the inner surface of the outer tube; and a tampon.

13. A method for manufacturing the tampon applicator as defined in claim 1, said method comprising a step specifically adapted for forming, such as moulding, said applicator, outer tube or assembly.

14. (canceled)

15. (canceled)

16. A method for manufacturing the assembly as defined in claim 12, said method comprising a step specifically adapted for forming, such as moulding, said applicator, outer tube or assembly.
